(19) United States
(12) Patent Application Publication
(10) Pub. No.: US 2020/0035360 A1
(43) Pub. Date: Jan. 30, 2020

(54) PREDICTIVE MODELING FOR HEALTH SERVICES

(71) Applicant: THE TRUSTEES OF INDIANA UNIVERSITY, Indianapolis, IN (US)

(72) Inventors: Joshua Ryan VEST, Carmel, IN (US); Nir MENACHEMI, Zionsville, IN (US); Shaun Jason GRANNIS, Indianapolis, IN (US); Paul Kenneth HALVERSON, Indianapolis, IN (US); Suranga Nath KASTHURIRATHNE, Bloomington, IN (US)

(21) Appl. No.: 16/523,176

(22) Filed: Jul. 26, 2019

Related U.S. Application Data
(60) Provisional application No. 62/711,182, filed on Jul. 27, 2018.

(51) Int. Cl. G16H 50/20; G06N 20/00; G16H 50/30

(52) U.S. Cl. CPC G16H 50/20; G16H 50/30; G06N 20/00

(57) ABSTRACT

Technologies for predicting need for one or more treatment services include obtaining data representative of a patient cohort. From the data, one or more features associated with each of the patients of the cohort are extracted. The one or more features includes features indicative of social determinants of each of the plurality of patients and of a general population of individuals. A predictive model for determining a need for referring a patient to the one or more treatment services is trained.

### CROSS-REFERENCE TO RELATED APPLICATIONS

The present disclosure claims the benefit of U.S. Provisional Patent Application Ser. No. 62/711,182, entitled “Predictive Modeling for Health Services,” filed Jul. 27, 2018, which is incorporated herein by reference in its entirety.

### BACKGROUND

The social determinants of health include a variety of behaviors, social situations, socioeconomic conditions, and physical and policy environments that contribute to health and well-being of individuals. These factors may increase patient complexity, complicate the delivery of care, and impact overall health outcomes. Health care delivery organizations, providers, and payers are becoming more attentive to the challenges and costs posed by patients' social determinants. For example, some health systems are partnering with community organizations to offer social services, and the American Medical Association endorses physician training in social determinants. Additionally, intervening on social determinants is a critical component of the Center for Medicare & Medicaid Services Accountable Health Communities program.

However, social determinants are infrequently screened, assessed, or addressed in primary care settings due a combination of factors. Providers may simply lack time or be insufficiently knowledgeable about social determinants. Even when aware of social determinants, providers may have concerns that these determinants cannot be adequately resolved in an office visit. Moreover, practices are hampered by insufficient documentation capability within electronic health records (EHRs). In addition, patients may be reluctant to share personal information. As such, patients' needs may be frequently unknown, underestimated, and left unmet. Standardized and systematic approaches to identifying those with social determinant needs may better support primary care workflows and the linkage of patients to necessary services.

### SUMMARY

One embodiment presented herein discloses a method for predicting need for one or more treatment services. The method generally includes obtaining data indicative of a plurality of patients. The method also includes extracting, from the data, one or more features associated with each of the plurality of patients. The one or more features including features indicative of social determinants of each of the plurality of patients and of a general population of individuals. The method also generally includes training, as a function of the extracted features, a predictive model for determining a need for referring a patient to the one or more treatment services.

Another embodiment presented herein discloses a computing server including one or more processors and a memory. The memory stores program code, which, when executed on the one or more processors, performs an operation for predicting need for one or more treatment services. The operation itself generally includes obtaining data indicative of a plurality of patients. The operation also includes extracting, from the data, one or more features associated with each of the plurality of patients. The one or more features including features indicative of social determinants of each of the plurality of patients and of a general population of individuals. The operation also generally includes training, as a function of the extracted features, a predictive model for determining a need for referring a patient to the one or more treatment services.

Yet another embodiment presented herein discloses one or more machine-readable storage media storing instructions, which, when executed, performs an operation for predicting need for one or more treatment services. The operation itself generally includes obtaining data indicative of a plurality of patients. The operation also includes extracting, from the data, one or more features associated with each of the plurality of patients. The one or more features including features indicative of social determinants of each of the plurality of patients and of a general population of individuals. The operation also generally includes training, as a function of the extracted features, a predictive model for determining a need for referring a patient to the one or more treatment services.

### BRIEF DESCRIPTION OF THE DRAWINGS

FIG. 1 illustrates an example of at least one embodiment of a computing environment for generating models for predicting a need for referring a patient to a social service;

FIG. 2 illustrates an example of at least one embodiment of a computing server described relative to FIG. 1 for generating models for predicting a need for referring a patient to a social service;

FIG. 3 illustrates an example of at least one embodiment of a method for generating a predictive model for predicting a need for referring a patient to a social service;

FIG. 4 illustrates an example of at least one embodiment of a method for predicting a need for referring a patient to a social service;

FIG. 5 illustrates an example graph representing sensitivity, specificity, accuracy, and positive predicted value (PPV) of clinical and master data vector models for any referrals, according to at least one embodiment; and

FIG. 6 illustrates an example graph representing sensitivity, specificity, accuracy, and PPV of both the clinical and master data vector models for different referral types.

### DESCRIPTION OF THE ILLUSTRATIVE EMBODIMENTS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

An increasing availability of diverse data sources has a potential to better inform health services delivery and health system performance. The widespread adoption of Electronic Health Records (EHRs) has increased the volume of electronically captured clinical data. Additionally, the growing use of interoperable systems and health information exchange promote access to more actionable information across different systems. Moreover, a growing number of social determinants of health (SDH) datasets describing social, physical, and policy environments in communities may be integrated with clinical information to augment overall data utility. Predictive modeling is one area of health care where emerging non-clinical data sources can be leveraged and commonly supports organizational planning, intervention allocation, risk adjustment, research, and health policy. Also, predictive models that include broader measures of patient's social determinants of health (SDH) may help align precision medicine and population health approaches for improving health.

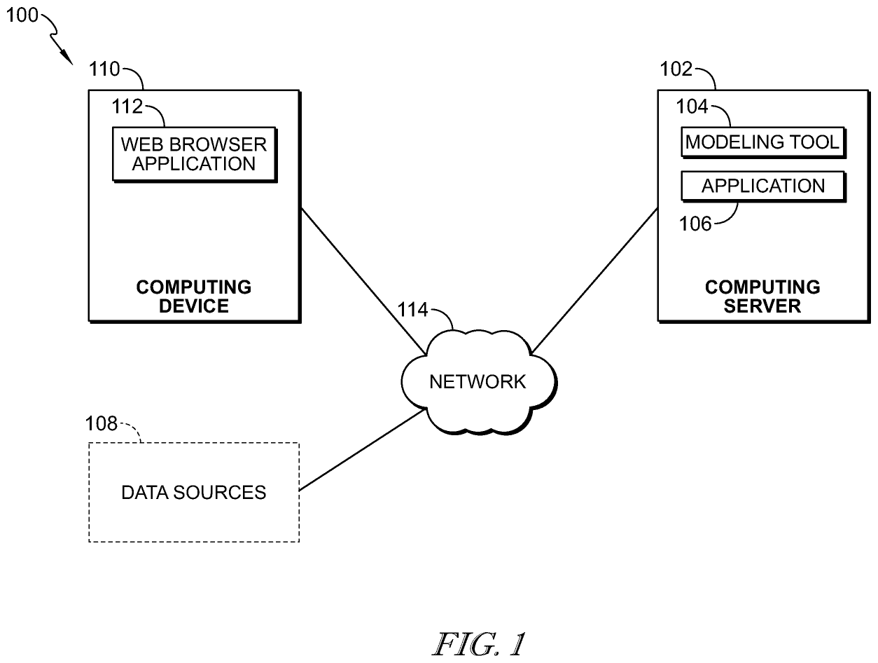

Referring now to FIG. 1, a computing environment 100 is shown for generating predictive models to determine a need for referral for a patient to one or more social services (also referred to herein as “treatment services” such as mental health, dietitian, social work, or other social determinants of health services) is shown. As shown, the computing environment 100 includes a computing server 102, one or more data sources 108, and a computing device 110, each interconnected with one another via a network 114 (e.g., the Internet).

The computing server 102 may be representative of a physical computing system (e.g., a desktop computer, a workstation, a laptop, etc.) or a virtual computing instance (e.g., in a cloud provider network). The illustrative computing server 102 includes a modeling tool 104 and an application 106. The modeling tool 104 may obtain data from a variety of data sources 108. The data sources 108 can include health system databases, records databases, or any location in which patient-related data (e.g., clinical, visit, medication, and population-level data) may be obtained. Particularly, the data sources 108 represent clinical, socioeconomic, and public health data sources to be evaluated to predict the need of various social service referrals among patients. From the data, the modeling tool 104 may build various predictive models (e.g., a decision model using only clinical data and another decision model using both clinical and SDH determinants) to assess the impact of SDH in improving performance. Moreover, contributions of these data on outcome of referrals to social services that inherently address patient's SDH (e.g., dietetics, social work, mental health) can also evaluated through the predictive models. A focus on referrals to such services may be relevant, given these services are intended to directly address the risk factors represented by many nonclinical data sources.

The data may provide features that the modeling tool 104, through a variety of feature extraction methods, may identify. Particularly, the modeling tool 104 may extract features relating to multiple social determinants of health As further described herein, the modeling tool 104, using the extracted features, train a predictive model used to evaluate, for a patient, a need for referral to one or more treatment services, such as behavioral health services, dietician counseling service, or social work service.

In the illustrative embodiment, the predictive models are configured to provide one or more social services that are predicted to be needed or recommended for the patient based on the patient's clinical and non-clinical (e.g., social determinants) data. To provide such recommendations, in some embodiments, the predictive models may be accessed by an application on a patient's device (e.g., the web browser application 112 on computing device 110). The application 106 may provide predicted need for one or more social services based on patient's relevant clinical, environmental, and behavioral data (e.g., provided by the patient via the web browser application 112). The application 106 is configured to provide a personalized list of “wrap-around” services that may benefit the patient, adding in additional details describing the benefit of such services, and how to access services in their community. It should be appreciated that, in some embodiments, the application 106 may be communicatively coupled to a hospital's health network such that the patient data may upload patient's information to the electronic health records (EHRs) or downloaded patient's clinical data from the electronic health records (EHRs) to continually or periodically updating the predictive models to provide accurate recommendations.

In such a case, the web browser application 112 may provide patient data to the modeling tool 104 via the application 106. In response, the predictive model may output predictive risk scores indicative of a probability of the patient needing a referral to a given treatment service. If one or more of the risk scores exceed a given threshold, the application 106 may generate an action to perform based on the result, such as generating a recommendation for a treatment service based on the score.

Advantageously, including patient- and population-level social determinant features and additional analytical methods for developing the predictive models addresses limitations in previous approaches, such as in performance of the models and sensitivity with respect to one or more of the treatment services.

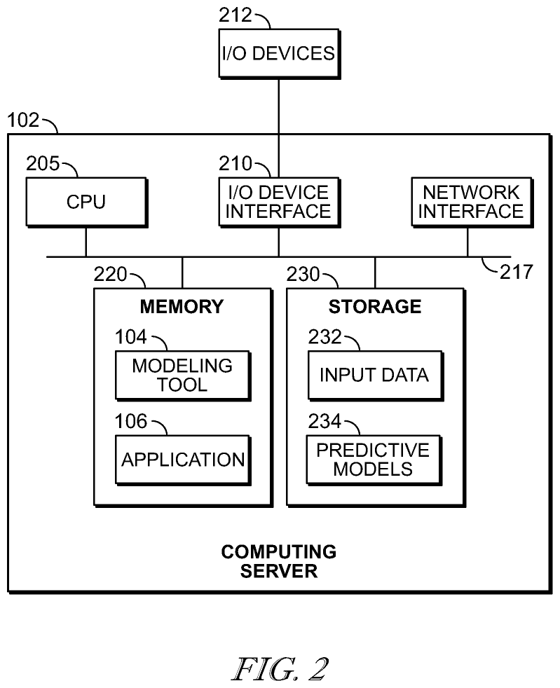

Referring now to FIG. 2, the computing server 102 may include, without limitation, a central processing unit (CPU) 205, an I/O device interface 210, a network interface 215, a memory 220, and a storage 230, each interconnected via an interconnect bus 217. Note, CPU 205 is included to be representative of a single CPU, multiple CPUs, a single CPU having multiple processing cores, and the like. Memory 220 is generally included to be representative of a random access memory. Storage 230 may be a disk drive storage device. Although shown as a single unit, storage 230 may be a combination of fixed and/or removable storage devices, such as fixed disc drives, removable memory cards, or optical storage, network attached storage (NAS), or a storage area network (SAN). The I/O device interface 210 may provide a communications interface between the computing server 102 and I/O devices 212. The I/O devices 212 may be embodied as any type of input/output device connected with or provided as a component to the computing server 102. Example I/O devices 212 include a keyboard, mouse, sensors, diagnostic equipment, speakers, interface devices, and other types of peripherals. The network interface 215 may be embodied as any communication circuit, device, or combination thereof, capable of enabling communications over a network between the computing server 102 and other devices (e.g., the client device 110). The network interface 215 may be configured to use any one or more communication technologies (e.g., wired or wireless communications) and associated protocols (e.g., Ethernet, Bluetooth, WiFi, etc.) to perform such communication.

Illustratively, the memory 220 includes the modeling tool 104 and the application 106 discussed relative to FIG. 1. The storage 230 includes input data 232 and predictive models 234. The input data 232 may be representative of patient information that is received from a remote device, such as the computing device 110. The computing server 102 may temporarily store the input data 232 in the storage 230 and provide the input data 232 to the predictive models 234. In turn, the predictive models 234 may generate risk scores indicative of whether to refer the patient to one or more treatment services.

Note, although FIG. 2 depicts the modeling tool 104, application 106, input data 232, and the predictive models as being included in a single computing server 102, one of skill in the art will recognize that each of these components may be configured in separate computing servers 102 or in different combinations. For example, the modeling tool 104 and predictive models 234 may reside in a given server, and the application 106 may reside on another server.

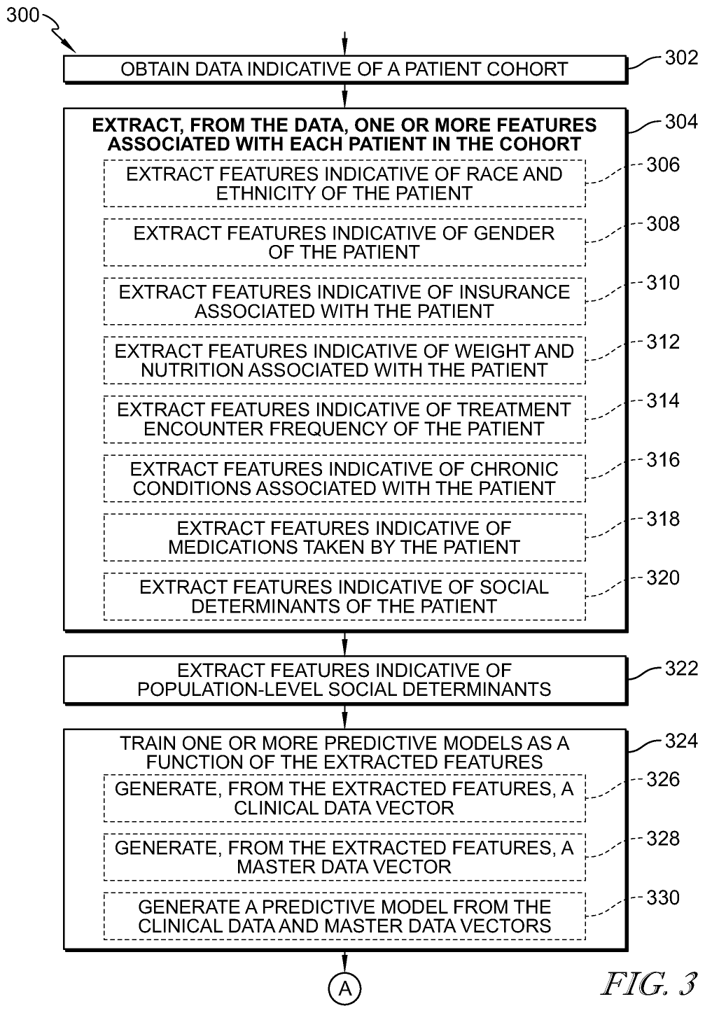

Referring now to FIG. 3, the computing server 102, in operation, may perform a method 300 for generating a predictive model for determining whether to refer a patient to a social service for treatment. As shown, the method 300 begins in block 302, in which the computing server 102 (e.g., via the modeling tool 104) obtains data indicative of a plurality of patients (also referred to herein as a “patient cohort”). In some embodiments, the computing server 102 obtains data that has occurred at least twenty-four hours prior to a final outcome of interest.

In block 304, the computing server 102 extracts, from the data, one or more features associated with each patient in the cohort. Various features from the data of each patient are extractable. For example, in block 306, the computing server 102 may extract features indicative of race and ethnicity associated with a given patient. As another example, in block 308, the computing server 102 may extract features associated with the gender of the patient. As yet another example, in block 310, the computing server 102 may extract features associated with insurance information of the patient. Further, in block 312, the computing server 102 may extract features indicative of the weight of the patient and nutrition adhered to by the patient. As another example, in block 314, the computing server 102 may extract features indicative of the treatment encounter frequency (e.g., an amount of visits by the patient to one or more treatment centers for a treatment service) associated with the patient. Even further, in block 316, the computing server 102 may extract features indicative of chronic conditions associated with the patient. As yet another example, in block 318, the computing server 102 may extract features indicative of medications taken by the patient. Further, in block 320, the computing server 102 may extract features indicative of social determinants of health (such as those described herein) associated with the patient.

In addition, in block 322, the computing server 102 extracts features indicative of population-level (e.g., in the aggregate, such as that provided by a clinical framework) social determinants of health. Such features can include socio-economic status, disease prevalence, and other miscellaneous factors (e.g., information on calls seeking public assistance).

In block 324, the computing server 102 generates and trains, from the extracted features, one or more predictive models. For instance, to do so, in block 326, the computing server 102 may generate, from the extracted features, a clinical data vector that includes patient-level data elements. In block 328, the computing server 102 may generate, from the extracted features, a master data vector that includes patient-level data and population-level social determinant features. In block 330, the computing server 102 may generate the predictive model from the clinical data and the master data vectors. For example, to do so, the computing server 102 may apply feature selection techniques (e.g., randomized LASSO feature selection) and machine learning algorithms to build classification models.

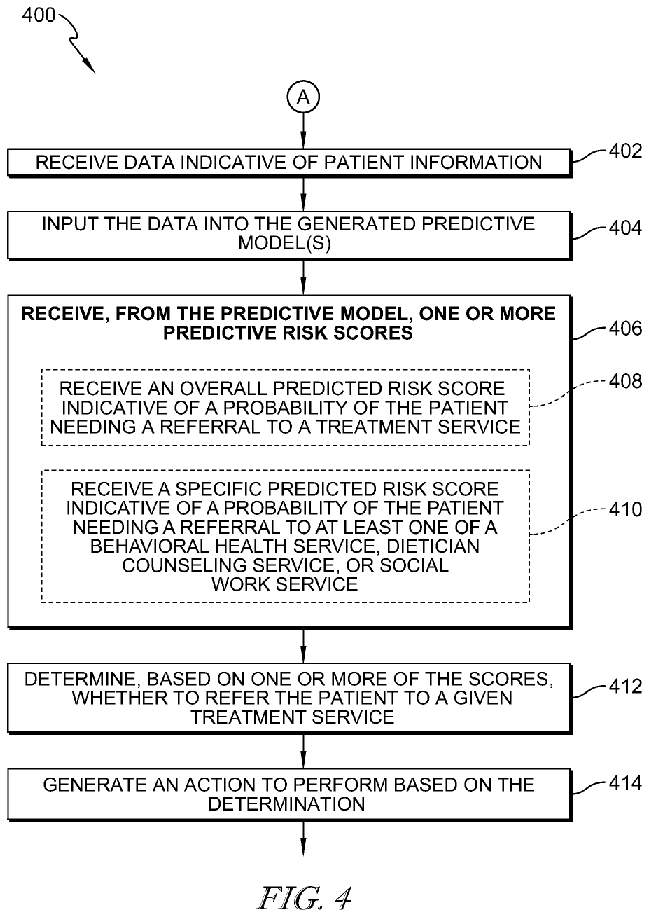

Referring now to FIG. 4, the computing server 102, in operation, may perform a method 400 for determining a need for referral of a patient to one or more treatment services based on patient information provided to the generated predictive models. As shown, the method 400 begins in block 402, in which the computing server 102 (e.g., via the application 106) receives data indicative of patient information. For example, the data received may originate from a remote device, such as a mobile device accessing the computing server 102 using a web browser application or mobile application. In block 404, the computing server 102 inputs the data into the predictive models generated from the data obtained from the data sources 108.

In block 406, the computing server 102 receives, one or more predictive risk scores determined by the predictive models. For instance, in block 408, the computing server 102 may receive an overall predictive risk score. The overall predictive risk score is indicative of a probability of the patient needing referral to any treatment service. In addition, in block 410, the computing server 102 receives a specific predictive risk score indicative of a patient needing a referral to a specific service. For example, this may include a behavioral health service, a dietician counseling service, or a social work service.

In block 412, the computing server 102 determines, based on the one or more predictive risk scores, whether to refer the patient to a given treatment service. For example, in some cases, each predictive risk score may correspond to a respective social service. If a given predictive risk score exceeds a particular threshold, the computing server 102 may determine that the patient should be referred to that corresponding service. In block 414, the computing server 102 generates an action to perform based on the determination. For example, assume the computing server 102 determines that the patient is to be referred to a behavioral health service. In such a case, the computing server 102 may identify one or more behavioral health services relative to a location of the patient and generate a report including the identified behavioral health services.

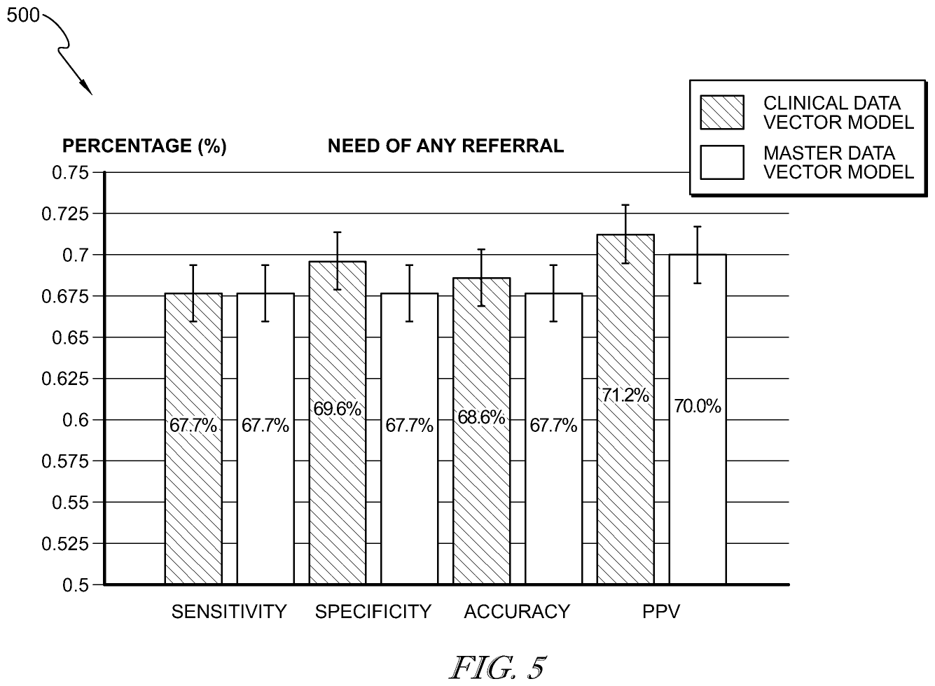

Referring now to FIG. 5, an example graph 500 representing sensitivity, specificity, accuracy, and positive predicted value (PPV) of clinical and master data vector models for any referrals generated by the computing server 102 are shown. In this example, the graph is based on patient data of a population of 84,317 adult patients (>18 years old) who had at least one outpatient visit between 2011-2016 collected from a healthcare system. The patient sample includes an adult, urban, primary care population: predominately female (64.9%), ethnically diverse (only 1 out of 4 patients was White, non-Hispanic), and with high chronic disease burdens.

As discussed above, the predictive models are configured to predict need for referrals to any social service overall, for referrals to individual SDH services, and also the union of all services. Such SDH services include mental health services, dietitian counseling, social work services, and all other social services, such as respiratory therapy, financial planning, medical legal partnership assistance, patient navigation, and/or pharmacist consultation.

From the EHR and INPC data, patient diagnoses (e.g., ICD-9 and ICD-10 codes), patient demographics (e.g., age, race/ethnicity, and gender), and counts of healthcare encounters were abstracted. The dataset covered all healthcare visits captured by the INPC. For decision modeling purposes, the data were processed as follows: (a) Diagnoses were reduced to binary indicators (present, absent) for the 20 most common chronic conditions and tobacco use. Charlson comorbidity index scores were also calculated for each patient using diagnosis codes; (b) Race and ethnicity were coded as series of mutually-exclusive binary indicators for Hispanic, African American, White (non-Hispanic), other, and unknown. Gender was expressed as a binary indicator. Patient age was determined at the study period midpoint and included as an integer variable; (c) Encounter frequency was reported as counts stratified by outpatient visits, emergency department encounters, and inpatient admissions.

This approach to structuring clinical diagnosis, demographic, and encounter data yielded 41 features, which comprised a clinical data vector. A total of 48 socioeconomic and public health indicators were selected to represent economic stability, neighborhood and physical environment, education, food, community and social context, and healthcare system. Due to the high variability among distributions for each feature, the sizes of the bins was determined by the Sturges rule. The master data vector is comprised of both the clinical data vector and the aforementioned social determinants.

The patient populations of each data vector was divided into two randomly selected groups of 90% of the patient population (i.e., training data) and 10% of the patient population (i.e., test data). Low prevalence of any outcome may produce an imbalanced data set and therefore may negatively impact decision model performance. As such, in the illustrative embodiment, the 90% training dataset was oversampled using the Synthetic Minority Over-sampling Technique (SMOTE).

Specifically, each training dataset was used to build a predictive model using the random forest classification algorithm. Random forest classification algorithm is configured to track record in healthcare decision making applications and perform internal feature selection. A total of 10 decision models for 10 different datasets (2 data vectors*5 outcomes of interest) was configured. In the illustrative embodiment, data cleaning, decision model development, and testing were performed using python and scikit-learn software. However, it should be appreciated that similar coding software may be used to implement the random forest classification algorithm to develop decision models. Additionally, it should be further appreciated that, in some embodiments, any machine learning technique may be used to construct the predictive models.

As described above, the performance of each decision model was assessed using the 10% test dataset. For each outcome under test, a paired sample t-test was used to compare the performance of decision models built using clinical and master datasets. For each record in the 10% test dataset, each decision model produced a predicted outcome (e.g., needs referral or does not need referral) with a predicted probability score. Additionally, optimal sensitivity and specificity scores for each decision model were determined using Youden indexes. Specifically, sensitivity, specificity, accuracy, positive predictive value (PPV), and area under the curve (ROC) for each of the models were determined with 95% confidence intervals (CI).

In this example, the majority of patients (53.07%) were referred to at least one social service. The most commonly referred service was dietitian (32.57%) followed by mental health services (18.51%) and social work (8.69%). Approximately one in five patients were referred to at least one of the remaining low prevalence services (i.e., other miscellaneous services), which include respiratory therapy, financial planning, medical legal partnership assistance, patient navigation, and/or pharmacist consultation. It should be noted that, using the aforementioned SMOTE technique, the rate of social work referrals increased in the training dataset from 8.69% to 16.39% prevalence to address data imbalance for decision model building.

Further, in this example, the decision model built using the clinical data vector demonstrated useful discriminating power with an Area Unver the Curve (AUC) value of 0.7454 for any referrals. As can be seen in the graph 500, this clinical data model had a sensitivity of 67.6%, specificity of 69.6%, accuracy of 68.6%, PPV of 71.2%. In comparison, the decision model that was built using the master data vector which included socioeconomic and public health features reported a sensitivity of 67.7%, specificity of 67.7%, accuracy of 67.7%, PPV of 70.0%, and an AUC value of 0.741. These measures did not differ significantly from those produced by the clinical data vector model (p>0.05), as evidenced by the overlapping 95% confidence intervals.

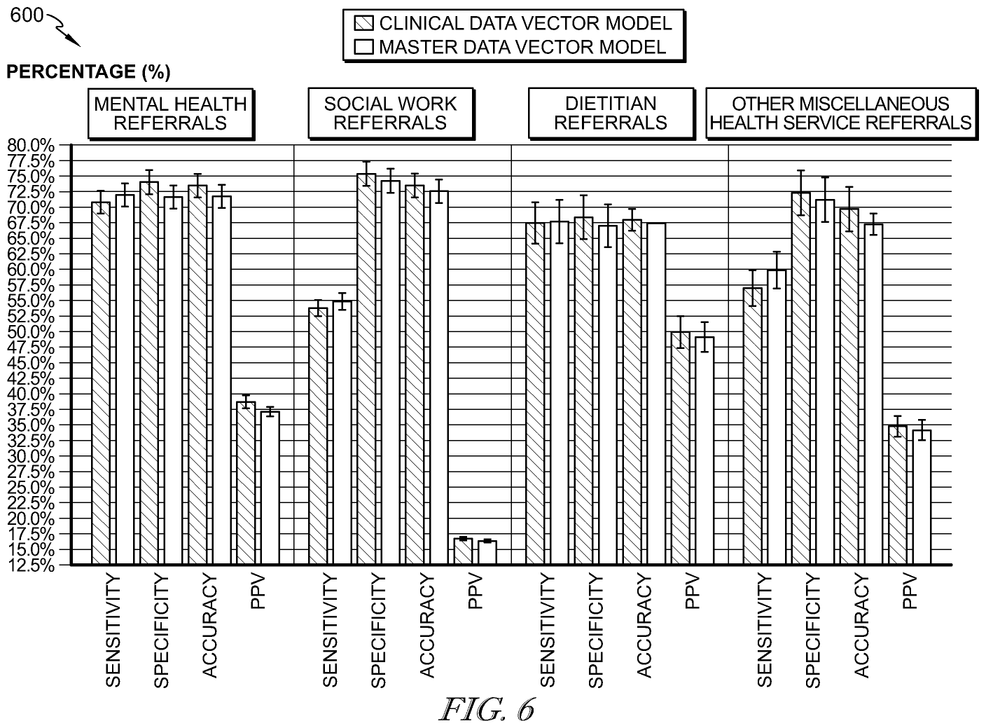

Further, the decision model built using the clinical data vector predicted need of mental health referrals with an AUC of 0.785. Referring now to an example graph 600 in FIG. 6, this model reported a sensitivity of 70.7%, specificity of 74.0%, and accuracy of 73.4%. In comparison, the master data vector model reported an AUC of 0.778, sensitivity of 71.9%, specificity of 71.7%, and accuracy of 71.7%. Both models produced comparatively low, although statistically similar PPV measures; 38.6% using clinical data only, and 37.0% using the master data vector.

In predicting social work referrals, the clinical data vector model demonstrated a sensitivity of 53.6%, specificity of 75.3%, accuracy of 73.5%, and PPV of 16.6%. In comparison, the master data vector reported a sensitivity of 53.6%, specificity of 74.1%, accuracy of 72.5%, and PPV of 16.6%. The clinical data model reported an AUC value of 0.731, while the master data model reported an AUC value of 0.713. It should be noted that the sensitivity and PPV values reported by both models were considerably smaller than other models. Additionally, both sensitivity measures reported large 95% confidence intervals (CI).

Additionally, the clinical data vector model for predicting need of dietitian referrals reported a sensitivity of 67.3%, specificity of 68.3%, accuracy of 67.9%, PPV of 49.9%, and an AUC of 0.743, while the decision model built using the master data vector reported a sensitivity of 67.3%, specificity of 66.9%, accuracy of 67.2, PPV of 49%, and an AUC of 0.73. As evidenced by the overlapping 95% confidence intervals, there was no statistically significant difference across any of the performance metrics reported across both models.

Moreover, the clinical data vector model for predicting other miscellaneous health service referrals demonstrated useful explanatory power with an AUC value of 0.711. As shown in example graph 600, this clinical data model had a sensitivity of 56.8%, specificity of 72.2%, accuracy of 69.6%, and PPV of 34.7%. In comparison, the master data vector model reported an AUC value of 0.708, sensitivity of 59.7%, specificity of 71.2%, accuracy of 68.9%, and PPV of 34.1%. Again, none of these measures presented significant difference between the two models.

In other words, the two predictive models shown in FIG. 6 have similar performance metrics, with overlapping 95% confidence intervals. However, PPV scores were less accurate for several outcomes. While decision models predicting need of any service exhibited PPV values greater than 65%, similar models predicting need of individual services yielded PPV's below 40%. Smaller PPV values may be attributed to the low rate of some services referrals. Because PPV evaluates the probability that a subject truly needs a service after having been predicted to need the service, risk prediction is more suitable for more prevalent outcomes of interest.

Predicting the need for social services referrals is responsive to recent calls for analytics that better match patients to services based on need, and also to match patients to services that address the upstream determinants of health. More importantly, services including social work, mental health, dietitian counseling, medical-legal partnerships, and others are of growing importance to health care organizations that, under changing reimbursement policies, are incentivized to prevent illness and promote health. The services delivered by these professionals directly address the determinants of health and support prevention activities. Because physicians are not trained to provide these services, patient receipt of these services depends on referrals to partner organizations or other care team members. Accurate stratification by risk is critical to efficiently and effectively delivering such services. Based on the predicted need for one or more social services, one or more referral requests may be automatically submitted to a corresponding service center to trigger a more efficient automated referral to specific services. In some embodiments, the predicted need for one or more social services may be relayed to employers such that the employees may optimize employee health and determine potential expenses for providing services to employees.

It should be appreciated that, in some embodiments, a granularity of a feature vector may be increased by tabulating counts of referral types for each patient to increase the model performance. Additionally, in some embodiments, missed appointments and/or time duration between initiating a referral and the occurrence of an encounter event such as a documented visit or a missed appointment may be considered to increase the model performance.

1. A method for predicting need for one or more treatment services, the method comprising:
obtaining data representative of a plurality of patients;
extracting, from the data, one or more features associated with each of the plurality of patients, the one or more features including features indicative of social determinants of each of the plurality of patients and of a general population of individuals; and
training, as a function of the extracted features, a predictive model for determining a need for referring a patient to the one or more treatment services.

2. The method of claim 1, wherein training the predictive model comprises generating, from the extracted features, a clinical data vector and a master data vector.

3. The method of claim 2, wherein training the predictive model further comprises generating the predictive model from the clinical data vector and a master data vector.

4. The method of claim 1, wherein extracting the one or more features comprises extracting features indicative of at least one of a race and ethnicity, gender, insurance, weight and nutrition, treatment encounter frequency, chronic conditions, or medications associated with each of the plurality of patients.

5. The method of claim 1, further comprising:
receiving data indicative of information associated with a first patient;
inputting the data into the predictive model; and
receiving, as a function inputting the data into the predictive model, one or more predictive risk scores.

6. The method of claim 5, further comprising:
determining, as a function of the one or more of the predictive risk scores, whether to refer a patient to one of the one or more treatment services; and
generate, in response to the determination, an action to perform.

7. The method of claim 5, wherein receiving the one or more predictive risk scores comprises receiving an overall predictive risk score indicative of a probability of the first patient needing a referral to a treatment service.

8. The method of claim 5, wherein receiving the one or more predictive risk scores comprises receiving a predictive risk score indicative of a probability of the first patient needing a referral to at least one of a behavioral health service, dietician counseling service, or social work service.

9. A computing server comprising:
one or more processors; and
a memory storing program code, which, when executed on the one or more processors, performs an operation for predicting need for one or more treatment services, the operation comprising:
obtaining data representative of a plurality of patients,
extracting, from the data, one or more features associated with each of the plurality of patients, the one or more features including features indicative of social determinants of each of the plurality of patients and of a general population of individuals, and
training, as a function of the extracted features, a predictive model for determining a need for referring a patient to the one or more treatment services.

10. The computing server of claim 9, wherein training the predictive model comprises generating, from the extracted features, a clinical data vector and a master data vector.

11. The computing server of claim 10, wherein training the predictive model further comprises generating the predictive model from the clinical data vector and a master data vector.

12. The computing server of claim 9, wherein extracting the one or more features comprises extracting features indicative of at least one of a race and ethnicity, gender, insurance, weight and nutrition, treatment encounter frequency, chronic conditions, or medications associated with each of the plurality of patients.

13. The computing server of claim 9, wherein the operation further comprises:
receiving data indicative of information associated with a first patient;
inputting the data into the predictive model; and
receiving, as a function inputting the data into the predictive model, one or more predictive risk scores.

14. The computing server of claim 13, wherein the operation further comprises:
determining, as a function of the one or more of the predictive risk scores, whether to refer a patient to one of the one or more treatment services; and
generate, in response to the determination, an action to perform.

15. The computing server of claim 13, wherein receiving the one or more predictive risk scores comprises receiving an overall predictive risk score indicative of a probability of the first patient needing a referral to a treatment service.

16. The computing server of claim 13, wherein receiving the one or more predictive risk scores comprises receiving a predictive risk score indicative of a probability of the first patient needing a referral to at least one of a behavioral health service, dietician counseling service, or social work service.

17. One or more machine-readable storage media storing a plurality of instructions, which, when executed, perform an operation for predicting need for one or more treatment services, the operation comprising:
obtaining data representative of a plurality of patients;
extracting, from the data, one or more features associated with each of the plurality of patients, the one or more features including features indicative of social determinants of each of the plurality of patients and of a general population of individuals; and
training, as a function of the extracted features, a predictive model for determining a need for referring a patient to the one or more treatment services.

18. The one or more machine-readable storage media of claim 17, wherein training the predictive model comprises:
generating, from the extracted features, a clinical data vector and a master data vector; and
generating the predictive model from the clinical data vector and a master data vector.

19. The one or more machine-readable storage media of claim 17, wherein extracting the one or more features comprises extracting features indicative of at least one of a race and ethnicity, gender, insurance, weight and nutrition, treatment encounter frequency, chronic conditions, or medications associated with each of the plurality of patients.

20. The one or more machine-readable storage media of claim 17, wherein the operation further comprises:
receiving data indicative of information associated with a first patient;
inputting the data into the predictive model; and
receiving, as a function inputting the data into the predictive model, one or more predictive risk scores.
